(19) United States
(12) Patent Application Publication
(10) Pub. No.: US 2021/0401855 A1
(43) Pub. Date: Dec. 30, 2021

(54) PHARMACEUTICAL FORMULATION CONTAINING COMBINATION OF M3 ANTAGONIST-BETA-2 AGONIST AND INHALED CORTICOSTEROIDS

(71) Applicants: Cai Gu Huang, Shrewsbury, MA (US); Abid Hussain, Shanghai (CN)

(72) Inventors: Cai Gu Huang, Shrewsbury, MA (US); Abid Hussain, Shanghai (CN)

(73) Assignee: Cai Gu Huang, Shrewsbury, MA (US)

(21) Appl. No.: 17/361,471

(22) Filed: Jun. 29, 2021

Related U.S. Application Data
(60) Provisional application No. 63/045,218, filed on Jun. 29, 2020.

(51) Int. Cl. A61K 31/569; A61K 31/4709; A61K 47/26; A61K 9/00; A61K 47/18

(52) U.S. Cl. CPC A61K 31/569; A61K 31/4709; A61K 47/183; A61K 9/0078; A61K 47/26

(57) ABSTRACT

The present invention relates to a pharmaceutical formulation and a method for administering the pharmaceutical formulation by nebulizing the pharmaceutical formulation with an inhaler. The propellant-free pharmaceutical formulation comprises: batefenterol or a pharmaceutically acceptable salt thereof, fluticasone furoate, and a pH adjusting agent.

### PRIORITY STATEMENT

This application claims the benefit of the filing date of U.S. Provisional Patent Application No. 63/045,218, filed on Jun. 29, 2020, which is incorporated herein by reference in its entirety.

### BACKGROUND OF THE INVENTION

Batefenterol, also known as 1-(2-{[2-chloro-4-({[(2R)-2-hydroxy-2-(8-hydroxy-2-oxo-1,2-dihydroquinolin-5-yl)ethyl]amino}methyl)-5-methoxyphenyl]carbamoyl} ethyl) piperidin-4-yl N-{[1,1′-biphenyl]-2-yl}carbamate; butanedioic acid, has the following chemical structure:

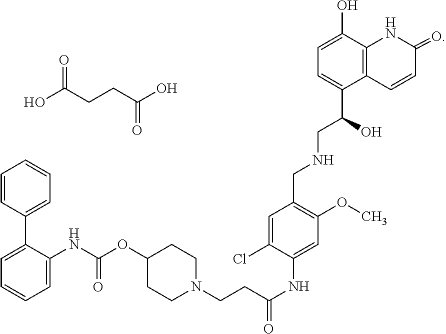 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12 ; O=C(O)CCC(=O)O]

Dual therapy using long-acting muscarinic antagonists (LAMAs) and long-acting b2-agonists (LABAs) in one inhaler produces superior bronchodilation to monotherapy with either class alone. Bifunctional compounds combining LAMA and LABA pharmacological actions, known as MABAs, are currently in clinical development, aiming to harness the synergy between these mechanisms of action and provide a potentially simpler technical and clinical development pathway than LAMA/LABA combination therapies. Batefenterol is a novel bi-functional molecule composed of both a muscarinic antagonist (MA) and a β2-agonist (BA) separated by an inert linker portion. The combination of an MA with a BA results in greater bronchodilation in the airways than either component alone.

Whereas, inhaled corticosteroids (ICS) are group of drugs such as fluticasone furoate and mometasone furoate monohydrate, that works as, efficient local anti-inflammatory effect, it can strengthen the stability of endotheliocyte, smooth muscle cell and lysosome membrane, Immunosuppression reaction and the synthesis of reduction antibody, thus the release of the activity media such as histamine is reduced and active reduction, and can alleviate antigen-antibody in conjunction with time the enzymatic processes that excites, suppress the synthesis of bronchoconstriction material and release and alleviate the contractile response of smooth muscle. Inhaled corticosteroid drugs work by reducing inflammation, swelling, and mucus production in the airways of a person with asthma. As a result, the airways are less inflamed and less likely to react to asthma triggers, allowing people with symptoms of asthma to have better control over their condition.

The combination of two compounds, selected as batefenterol and an ICS, have valuable pharmacological properties. Batefenterol and fluticasone furoate can provide therapeutic benefit in the treatment of asthma and chronic obstructive pulmonary disease.

Corticosteroid and combination therapy with a LABA/LABA is becoming an established method for the maintenance treatment of asthma and COPD. Moreover, a single compound with MABA activity, such as batefenterol, has advantages over the use of two separate compounds. LABA is long-acting beta agonist. MABA is muscarinic antagonist beta agonist. As a single pharmacokinetic (PK) profile exists for both pharmacological activities, there is the potential to maximize the synergy between the two mechanisms of action. The technical and clinical development pathway is also simpler for a single compound than for a co-formulation of two separate compounds.

### SUMMARY OF THE INVENTION

The present invention relates to pharmaceutical formulations of batefenterol and fluticasone furoate, and their pharmaceutically acceptable salts or solvates, which can be administered by a soft mist inhalation or nebulization inhalation method. The pharmaceutical formulations according to the invention meet high quality standards.

One aspect of the present invention is to provide a pharmaceutical formulation containing batefenterol and fluticasone furoate, and other inactive ingredients, which meets the high quality standards necessary to achieve optimum nebulization of a solution using a soft mist inhaler or nebulization inhaler. In one embodiment, the stability of the formulation is a storage time of few months or years, preferably 1-6 months, more preferably one year, and most preferably three years. In one embodiment, the combination formulation has a storage time of at least about 6 months at a temperature from about 15° C. to about 25° C. In one embodiment, the combination formulation has a storage time of at least about 1 year at a temperature from about 15° C. to about 25° C. In one embodiment, the combination formulation has a storage time of at least about 2 years at a temperature from about 15° C. to about 25° C. In one embodiment, the combination formulation has a storage time of at least about 3 years at a temperature from about 15° C. to about 25° C.

Another aspect of the current invention is to provide pharmaceutical formulations containing batefenterol and fluticasone furoate, which can be nebulized by an inhaler device, wherein the aerosol produced falls reproducibly within a specified range for particle size, such as less than about 10 μm.

Another aspect of the invention is to provide pharmaceutical formulations comprising batefenterol and fluticasone furoate, and other inactive excipients which can be administered by nebulization inhalation using an ultra-sonic based or air pressure based nebulizer/inhaler. In one embodiment, the stability of the formulation is a storage time of few months or years, preferably 1-6 months, more preferably one year, and most preferably three years. In one embodiment, the combination formulation has a storage time of at least about 6 months at a temperature from about 15° C. to about 25° C. In one embodiment, the combination formulation has a storage time of at least about 1 year at a temperature from about 15° C. to about 25° C. In one embodiment, the combination formulation has a storage time of at least about 2 years at a temperature from about 15° C. to about 25° C. In one embodiment, the combination formulation has a storage time of at least about 3 years at a temperature from about 15° C. to about 25° C.

Another aspect of the current invention is to provide stable pharmaceutical formulations containing batefenterol and fluticasone furoate and other excipients which can be administered by soft mist inhalation using an atomizer inhaler. The formulation have substantially long term stability. In one embodiment, the combination formulation has a storage time of at least about 6 months at a temperature from about 15° C. to about 25° C. In one embodiment, the combination formulation has a storage time of at least about 1 year at a temperature from about 15° C. to about 25° C. In one embodiment, the combination formulation has a storage time of at least about 2 years at a temperature from about 15° C. to about 25° C.

More specifically, another aspect of the current invention is to provide stable pharmaceutical formulations containing batefenterol and fluticasone furoate, and other excipients, which can be administered by nebulization inhalation using an ultrasonic jet or mesh nebulizer. The inventive formulation has substantially long term stability. The formulations may be a storage time of at least about 6-24 months at a temperature of from about 15° C. to about 25° C. In one embodiment, the combination formulation has a storage time of at least about 6 months at a temperature from about 15° C. to about 25° C. In one embodiment, the combination formulation has a storage time of at least about 1 year at a temperature from about 15° C. to about 25° C. In one embodiment, the combination formulation has a storage time of at least about 2 years at a temperature from about 15° C. to about 25° C.

### BRIEF DESCRIPTION OF THE DRAWINGS

FIG. 1 shows a longitudinal section through an atomizer in the stressed state.

FIG. 2 shows a counter element of the atomizer.

### DETAILED DESCRIPTION OF THE INVENTION

It is significant to achieve a better delivery of active substances to the lung for the treatment of lung diseases. It is important to increase the lung deposition of a drug delivered by an inhalation method.

There is a need in the art to improve the delivery of drugs by inhalation by significantly increasing lung deposition. The soft mist or nebulization inhalation device disclosed in US20190030268 can significantly increase the lung deposition of inhalable drugs.

Those inhalers can nebulize a small amount of a liquid formulation within a few seconds into an aerosol that is suitable for therapeutic inhalation. Those inhalers are particularly suitable for administering the liquid formulations of the invention.

In one embodiment, the soft mist devices used to administer the pharmaceutical formulation of the present invention are those in which an amount of less than about 70 microliters of pharmaceutical solution can be nebulized in one puff so that the inhalable part of aerosol corresponds to the therapeutically effective quantity. In one embodiment, the soft mist devices used to administer the pharmaceutical formulation of the present invention are those in which an amount of less than about 30 microliters of pharmaceutical solution can be nebulized in one puff so that the inhalable part of aerosol corresponds to the therapeutically effective quantity. In one embodiment, the soft mist devices used to administer the pharmaceutical formulation of the present invention are those in which an amount of less than about 15 microliters of pharmaceutical solution can be nebulized in one puff so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

In one embodiment, the nebulization devices used to administer the pharmaceutical formulations of the invention are those in which an amount of less than about 8 milliliters of the pharmaceutical solution can be nebulized in one puff so that the inhalable part of aerosol corresponds to the therapeutically effective quantity. In one embodiment, the nebulization devices used to administer the pharmaceutical formulations of the invention are those in which an amount of less than about 2 milliliters of the pharmaceutical solution can be nebulized in one puff so that the inhalable part of aerosol corresponds to the therapeutically effective quantity. In one embodiment, the nebulization devices used to administer the pharmaceutical formulations of the invention are those in which an amount of less than about 1 milliliter of the pharmaceutical solution can be nebulized in one puff so that the inhalable part of aerosol corresponds to the therapeutically effective quantity. In one embodiment, the average particle size of aerosol formed from one puff is less than about 15 microns. In one embodiment, the average particle size of aerosol formed from one puff is less than about 10 microns.

A device of this kind for the propellant-free administration of a metered amount of a liquid pharmaceutical composition for inhalation is described in detail in, for example, US20190030268, entitled “inhalation atomizer comprising a blocking function and a counter”.

The pharmaceutical formulation in the nebulizer is converted into aerosol destined for the lungs. The nebulizer uses high pressure to spray the pharmaceutical formulation.

The pharmaceutical formulation, which can be a solution, is stored in a reservoir in this kind of inhaler. The formulations must not contain any ingredients which might interact with the inhaler to affect the pharmaceutical quality of the solution or of the aerosol produced. In addition, the active substances in pharmaceutical formulations are very stable when stored and can be administered directly.

In one embodiment, the formulations, which can be solutions, for use with the inhaler described above contains additives, such as the disodium salt of edetic acid (sodium edetate), to reduce the incidence of spray anomalies and to stabilize the formulation. In one embodiment, the formulations of have a minimum concentration of sodium edetate.

One aspect of the present invention is to provide a pharmaceutical formulation containing batefenterol and fluticasone furoate and other excipients, which meet the high standards needed to achieve optimum nebulization of the formulation using a soft mist inhaler. In one embodiment, the stability of the formulation is a storage time of few months or years, preferably 1-6 months, more preferably one year, and most preferably three years. In one embodiment, the combination formulation has a storage time of at least about 6 months at a temperature from about 15° C. to about 25° C. In one embodiment, the combination formulation has a storage time of at least about 1 year at a temperature from about 15° C. to about 25° C. In one embodiment, the combination formulation has a storage time of at least about 2 years at a temperature from about 15° C. to about 25° C. In one embodiment, the combination formulation has a storage time of at least about 3 years at a temperature from about 15° C. to about 25° C.

The formulations according to the invention include as active substances batefenterol or its pharmaceutically acceptable salts or its active metabolites and an inhaled corticosteroid selected from fluticasone furoate or its pharmaceutically acceptable salt.

In one embodiment, the batefenterol and fluticasone furoate, or their pharmaceutically acceptable salts are dissolved in a solvent. In one embodiment, the solvent comprises water. In one embodiment, the solvent is water.

In one embodiment the MABA compound, specifically batefenterol, is administered by inhalation to deliver a dose ranging from about 1 mcg/daily to about 1000 mcg/daily. In one embodiment, the batefenterol is administered at a dose of about 100 mcg per day. In one embodiment, the batefenterol is administered at a dose of about 250 mcg per day. In one embodiment, the batefenterol is administered at a dose of about 500 mcg per day.

In one embodiment, the MABA compound, specifically batefenterol is administered by inhalation to deliver a dose of the free cation of about 15.625 mcg once or twice daily. In one embodiment, the MABA compound, specifically batefenterol, is administered by inhalation to deliver a dose of the free cation of about 31.25 mcg once or twice daily. In one embodiment, the MABA compound, specifically batefenterol is administered by inhalation to deliver a dose of the free cation of about 62.5 mcg once or twice daily. In one embodiment, the MABA compound, specifically batefenterol, is administered by inhalation to deliver a dose of the free cation of about 125 mcg once or twice daily. In one embodiment, the MABA compound, specifically batefenterol, is administered once-daily.

In one embodiment, the MABA compound, specifically batefenterol, is administered by inhalation, once daily, to deliver a dose of the free cation of about 15.625 mcg per day.

In one embodiment, the MABA compound, specifically batefenterol, is administered by inhalation, once daily, to deliver a dose of the free cation of about 31.25 mcg per day.

In one embodiment, the MABA compound, specifically batefenterol, is administered by inhalation, once daily, to deliver a dose of the free cation of about 62.5 mcg per day.

In one embodiment, the MABA compound, specifically batefenterol, is administered by inhalation, once daily, to deliver a dose of the free cation of about 125 mcg per day.

The chosen corticosteroid may be administered, for example, by inhalation at a dose of from about 1 mcg/day to about 1000 mcg/day (calculated as the free base). When the corticosteroid is fluticasone furoate it may be administered by inhalation at a dose ranging from about 25 mcg daily to about 800 mcg daily, and if necessary in divided doses. In one embodiment, the daily dose of fluticasone furoate is about 25 mcg. In one embodiment, the daily dose of fluticasone furoate is about 50 mcg. In one embodiment, the daily dose of fluticasone furoate is about 100 mcg. In one embodiment, the daily dose of fluticasone furoate is about 200 mcg. In one embodiment, the daily dose of fluticasone furoate is about 300 mcg. In one embodiment, the daily dose of fluticasone furoate is about 400 mcg. In one embodiment, the daily dose of fluticasone furoate is about 600 mcg. In one embodiment, the daily dose of fluticasone furoate is about 800 mcg. In one embodiment, the dose of fluticasone furoate is administered once-daily.

In one embodiment, the daily dose of fluticasone furoate is about 200 mcg. In one embodiment, the daily dose of budesonide is about 100 mcg. In one embodiment, the daily dose of fluticasone furoate is about 50 mcg.

In one embodiment, the corticosteroid is fluticasone furoate administered by inhalation at a dose ranging from about 100 mcg daily to about 500 mcg daily, and if necessary in divided doses. In one embodiment, the daily dose of fluticasone furoate is about 100 mcg. In one embodiment, the daily dose of fluticasone furoate is about 250 mcg. In one embodiment, the daily dose of fluticasone furoate is about 500 mcg.

In one embodiment, the present invention provides a pharmaceutical combination product for once-daily administration by inhalation, wherein the batefenterol is administered at a dose of the free cation of about 125 mcg per day, and the fluticasone furoate is administered at a dose of about 100 mcg per day.

In one, the present invention provides a pharmaceutical combination product for once-daily administration by inhalation, wherein the batefenterol is administered at a dose of the free cation of about 62.5 mcg per day, and fluticasone furoate is administered at a dose of about 100 mcg per day.

The concentration of the batefenterol or its pharmaceutically acceptable salt depends on the therapeutic effects. In one embodiment, the concentration of batefenterol or its pharmaceutically acceptable salts in the formulation ranges from about 1 mg/ml to about 200 mg/ml. In one embodiment, the concentration of batefenterol or its pharmaceutically acceptable salts in the formulation ranges from about 5 mg/ml to about 150 mg/ml. In one embodiment, the concentration of batefenterol or its pharmaceutically acceptable salts in the formulation ranges from about 10 mg/ml to about 30 mg/ml. In one embodiment, the soft mist devices used to administer the pharmaceutical formulation of the present invention atomizes about 10 microliters to about 15 microliters of the formulation about 10-15 times per use, so that the inhalable part of aerosol corresponds to the therapeutically effective quantity.

The concentration of the fluticasone furoate or its pharmaceutically acceptable salt depends on the therapeutic effects. In one embodiment, the concentration of fluticasone furoate or its pharmaceutically acceptable salts in the formulation ranges from about 100 mg/100 ml to about 20 g/100 ml. In one embodiment, the concentration of fluticasone furoate or its pharmaceutically acceptable salts in the formulation ranges from about 1.5 g/100 ml to about 19 g/100 ml. In one embodiment, the concentration of fluticasone furoate or its pharmaceutically acceptable salts in the formulation ranges from about 1.5 g/100 ml to about 2.5 g/100 ml. In one embodiment, the concentration of fluticasone furoate or its pharmaceutically acceptable salts in the formulation ranges from about 17 g/100 ml to about 19 g/100 ml. In one embodiment, the soft mist devices used to administer the pharmaceutical formulation of the present invention atomizes about 10 microliters to about 15 microliters of the formulation about 10-15 times per use, so that the inhalable part of aerosol corresponds to the therapeutically effective quantity.

In one embodiment, the formulations include an acid or base, as a pH adjusting agent. In one embodiment, the pH adjusting agent is selected from the group consisting of, hydrochloric acid, citric acid or its buffers, and/or the salts thereof.

Other comparable pH adjusting agents can also be used. Suitable pH adjusting agents include, but are not limited to, citric acid and sodium hydroxide.

Selecting the proper pH improves the stability of the formulation. In one embodiment, the pH of the formulation ranges from about 2.0 to about 6.0. In one embodiment, the pH of the formulation ranges from about 3.0 to about 5.0. In one embodiment, the pH of the formulation ranges from about 3.0 to about 4.0.

In one embodiment, the formulations include edetic acid (EDTA) or one of the known salts thereof, disodium edetate or edetate disodium dihydrate, as a stabilizer or complexing agent. In one embodiment, the formulation contains edetic acid and/or a salt thereof.

Other comparable stabilizers or complexing agents can be used. Examples of suitable stabilizers or complexing agents include, but are not limited to, citric acid, edetate disodium, and edetate disodium dihydrate.

The phrase “complexing agent,” as used herein, means a molecule which is capable of entering into complex bonds. Preferably, these compounds should have the effect of complexing cations. In one embodiment, the concentration of the stabilizer or complexing agent ranges from about 1 mg/100 ml to about 500 mg/100 ml. In one embodiment, the concentration of the stabilizer or complexing agent ranges from about 5 mg/100 ml to about 200 mg/100 ml. In one embodiment, the stabilizer or complexing agent is edetate disodium dihydrate at a concentration of about 10 mg/100 ml.

In one embodiment, all the ingredients of the formulation are present in solution.

The term “additive,” as used herein, means any pharmacologically acceptable and therapeutically useful substance which is not an active substance, but can be formulated together with the active substances in a pharmacologically suitable solvent, in order to improve the qualities of the formulation. Preferably, these substances have no pharmacological effects or no appreciable, or at least no undesirable, pharmacological effects in the context of the desired therapy.

Examples of additives include, but are not limited to, stabilizers, complexing agents, antioxidants, surfactants, and/or preservatives which prolong the shelf life of the finished pharmaceutical formulation, vitamins, and/or other additives known in the art.

In one embodiment, preservatives are added to the formulation to protect the formulation from contamination with pathogenic bacteria. Suitable preservatives include, but are not limited to, benzalkonium chloride, benzoic acid, and sodium benzoate. In one embodiment, the formulation contains only benzalkonium chloride. In one embodiment, the quantity of the preservative ranges from about 2 mg/100 ml to about 300 mg/100 ml. In one embodiment, the preservative is benzalkonium chloride in an amount of about 10 mg/100 ml.

In one embodiment, the formulations include a solubility enhancing agent, such as Tween 80, or a cyclodextrin derivative. In one embodiment, the solubility enhancing agent is Tween 80, or one of the known salts thereof.

In one embodiment, the formulations for soft mist inhalation include a surfactant or other solubility enhancing agent as a solubilizing agent. In one embodiment, the solubilizing agents is a surfactant. In one embodiment, the surfactant is selected from the group consisting of polysorbate, for example, polysorbate 20 and or polysorbate 80; poloxamer; SBECD; sodium dodecyl sulfate (SDS); sodium laurel sulfate; sodium octyl glycoside; polyethyl glycol; polypropyl glycol; and copolymers, or any mixture thereof. In one embodiment, the concentration of the surfactant ranges from about Omg/100 ml to about 100 mg/100 ml. In one embodiment, the concentration of the surfactant ranges from about 20 mg/100 ml to about 30 mg/100 ml.

Another aspect of the invention is to provide stable pharmaceutical soft mist formulations containing batefenterol and fluticasone furoate along with other excipients which can be administered by soft mist inhalation using an atomizer inhaler. In one embodiment, the formulation has substantially long term stability. In one embodiment, the formulation has a storage time of at least about 6 months at a temperature from about 15° C. to about 25° C. In one embodiment, the formulation has a storage time of at least about 1 year at a temperature from about 15° C. to about 25° C. In one embodiment, the formulation has a storage time of at least about 2 years at a temperature from about 15° C. to about 25° C. In one embodiment, the formulation has a storage time of at least about 3 years at a temperature from about 15° C. to about 25° C.

Another aspect of the invention is to provide pharmaceutical formulations of nebulization solutions comprising batefenterol and fluticasone furoate and other inactive excipients which can be administered by nebulization inhalation using an ultra-sonic based or air pressure based nebulizer/inhaler. In one embodiment, the stability of the formulation is a storage time of few months or years, preferably 1-6 months, more preferably one year, and most preferably three years. In one embodiment, the formulation has a storage time of at least about 6 months at a temperature from about 15° C. to about 25° C. In one embodiment, the formulation has a storage time of at least about 1 year at a temperature from about 15° C. to about 25° C. In one embodiment, the formulation has a storage time of at least about 2 years at a temperature from about 15° C. to about 25° C. In one embodiment, the formulation has a storage time of at least about 3 years at a temperature from about 15° C. to about 25° C.

Another aspect of the invention is to provide stable pharmaceutical formulations containing batefenterol and fluticasone furoate and other excipients which can be administered by nebulization inhalation using ultra-sonic based or air pressure based nebulizers/inhalers. In one embodiment, the formulations have substantially long term stability. The formulations may be a storage time of at least about 6-24 months at a temperature of from about 15° C. to about 25° C. In one embodiment, the formulation has a storage time of at least about 6 months at a temperature from about 15° C. to about 25° C. In one embodiment, the formulation has a storage time of at least about 1 year at a temperature from about 15° C. to about 25° C. In one embodiment, the formulation has a storage time of at least about 2 years at a temperature from about 15° C. to about 25° C.

In one embodiment, the formulations include sodium chloride. In one embodiment, the concentration of the sodium chloride ranges from about 0.3 g/100 ml to about 1.13 g/100 ml.

In one embodiment of the nebulization formulation, the active ingredient concentration of batefenterol and fluticasone furoate ranges from about 20 mg/4 ml to about 500 mg/4 ml. In one embodiment of the nebulization formulation, the active ingredient concentration of batefenterol and fluticasone furoate ranges from about 200 mg/4 ml to about 400 mg/4 ml. In one embodiment of the nebulization formulation, the active ingredient concentration of batefenterol and fluticasone furoate ranges from about 200 mg/4 ml to about 300 mg/4 ml.

In one embodiment, the nebulization formulations include a surfactant or other solubility enhancing agents as a solubilizing agent. In one embodiment, the solubilizing agents is a surfactant. In one embodiment, the surfactant is selected from the group consisting of polysorbate, for example, polysorbate 20 and, polysorbate 80; poloxamer; SBECD; sodium dodecyl sulfate (SDS); sodium laurel sulfate; sodium octyl glycoside; polyethyl glycol; polypropyl glycol; and copolymers, or any mixture thereof. In one embodiment, the concentration of the surfactant ranges from about Omg/100 ml to about 100 mg/100 ml. In one embodiment, the concentration of the surfactant ranges from about 20 mg/100 ml to about 30 mg/100 ml.

Another aspect of the invention is to provide stable pharmaceutical nebulization formulations containing batefenterol and fluticasone furoate and other excipients that can be administered by soft mist inhalation using an atomizer inhaler. In one embodiment, the formulation has substantially long term stability. ° C. In one embodiment, the formulation has a storage time of at least about 6 months at a temperature from about 15° C. to about 25° C. In one embodiment, the formulation has a storage time of at least about 1 year at a temperature from about 15° C. to about 25° C. In one embodiment, the formulation has a storage time of at least about 2 years at a temperature from about 15° C. to about 25° C.

Selecting the proper pH range provides improves stability of the nebulization formulation and maintains the solubility of the batefenterol and fluticasone furoate. The pH can be adjusted to the desired pH by adding an acid, e.g., HCl, or by adding a base, e.g., NaOH or by a combination of HCl and NaOH to achieve the desired buffer concentration and pH value.

In one embodiment, the pH value of the nebulization formulation ranges from about 3 to about 6. In one embodiment, the pH value of the nebulization formulation ranges from about 3 to about 5. In one embodiment, the pH value of the nebulization formulation ranges from about 3 to about 4.

In one embodiment, the nebulization formulations according to the present invention are filled into canisters to form a highly stable formulation for use in a nebulization device. The formulations exhibit substantially no particle growth, change of morphology, or precipitations. There is also no, or substantially no, problem of suspended particles being deposited on the surface of either canisters or valves, so that a dose of the formulations can be discharged from a suitable nebulization device with high dose uniformity. In one embodiment, the nebulizer is selected from an ultrasonic nebulizer, a jet nebulizer, or a mesh nebulizer, such as Pari eFlow nebulization inhaler, or other commercially available ultrasonic nebulizer, jet nebulizer or mesh nebulizer.

In one embodiment, the inhalation device is a soft mist inhaler. In one embodiment, to produce an aerosol of the pharmaceutical soft mist formulations containing batefenterol and fluticasone furoate, the formulations are administered using an inhaler of the kind described herein. Here we again expressly mention the patent documents described hereinbefore, to which reference is hereby made.

A soft mist inhaler device of this kind for the propellant-free administration of a metered amount of a liquid pharmaceutical composition for inhalation is described in detail in, for example, US20190030268, entitled “inhalation atomizer comprising a blocking function and a counter”.

The pharmaceutical formulation solution in the nebulizer is converted into aerosol destined for the lungs. The nebulizer uses high pressure to spray the pharmaceutical solution.

The soft mist inhalation device can be carried anywhere by the patient, since it has a cylindrical shape and a handy size of less than about 8 cm to 18 cm long and 2.5 cm to 5 cm wide. The nebulizer sprays a defined volume of the pharmaceutical formulation out through small nozzles at high pressures, so as to produce inhalable aerosols.

The preferred atomizer comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, and an inside part 17.

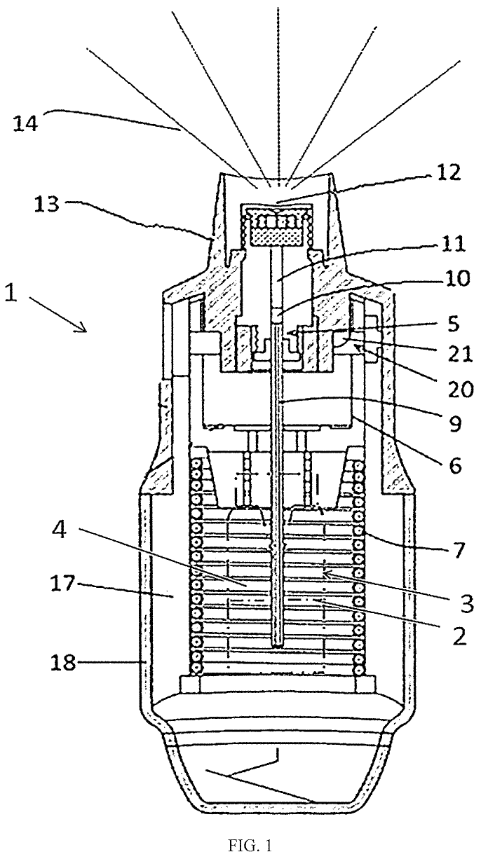

The inhalation atomizer 1 comprising the block function and the counter described above for spraying a medicament fluid 2 is depicted in FIG. 1 in a stressed state. The atomizer 1 comprising the block function and the counter described above is preferred as a portable inhaler and requires no propellant gas.

FIG. 1 shows a longitudinal section through the atomizer in the stressed state.

For the typical atomizer 1 comprising the block function and the counter described above, an aerosol 14 that can be inhaled by a patient is generated through the atomization of the fluid 2, which is preferably formulated as a medicament liquid. The medicament is typically administered at least once a day, more specifically multiple times a day, preferably at predetermined time gaps, according to how seriously the illness affects the patient.

In an embodiment, the atomizer 1 described above has substitutable and insertable vessel 3, which contains the medicament fluid 2. A reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 described above is in the vessel 3 to provide, e.g., up to 200 doses. A typical vessel 3 has a volume of about 2 ml to about 10 ml. A pressure generator 5 in the atomizer 1 is used to deliver and atomize the fluid 2 in a predetermined dosage amount. The fluid 2 can be released and sprayed in individual doses, specifically from about 5 to about 30 microliters.

In an embodiment, the atomizer 1 described above may have a pressure generator 5 and a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 can be separated from the atomizer 1 for substitution.

In an embodiment, when drive spring 7 is stressed in an axial direction, the delivering tube 9, the vessel 3 along with the holder 6 will be shifted downwards. Then the fluid 2 will be sucked into the pressure room 11 through delivering tube 9 and the non-return valve 10.

In one embodiment, after releasing the holder 6, the stress is eased. During this process, the delivering tube 9 and closed non-return valve 10 are shifted back upward by releasing the drive spring 7. Consequently, the fluid 2 is under pressure in the pressure room 11. The fluid 2 is then pushed through the nozzle 12 and atomized into an aerosol 14 by the pressure. A patient can inhale the aerosol 14 through the mouthpiece 13, while the air is sucked into the mouthpiece 13 through air inlets 15.

The inhalation atomizer 1 described above has an upper shell 16 and an inside part 17, which can be rotated relative to the upper shell 16. A lower shell 18 is manually operable to attach onto the inside part 17. The lower shell 18 can be separated from the atomizer 1 so that the vessel 3 can be substituted and inserted.

In one embodiment of the inhalation atomizer 1 described above has a lower shell 18, which carries the inside part 17, and is rotatable relative to the upper shell 16. As a result of rotation and engagement between the upper unit 17 and the holder 6, through a gear 20, the holder 6 is axially moved counter to the force of the drive spring 7, and the drive spring 7 is stressed.

In an embodiment, in the stressed state, the vessel 3 is shifted downwards and reaches a final position, which is demonstrated in FIG. 1. The drive spring 7 is stressed in this final position. Then the holder 6 is clasped. Therefore, the vessel 3 and the delivering tube 9 are prevented from moving upwards so that the drive spring 7 is stopped from easing.

In an embodiment, the atomizing process occurs after releasing the holder 6. The vessel 3, the delivering tube 9 and the holder 6 are shifted back by the drive spring 7 to the beginning position. This is referred to herein as major shifting. When major shifting occurs, the non-return valve 10 is closed and the fluid 2 is under pressure in the pressure room 11 by the delivering tube 9, and then the fluid 2 is pushed out and atomized by the pressure.

In an embodiment, the inhalation atomizer 1 described above may have a clamping function. During the clamping, the vessel 3 preferably performs a lifting shift for the withdrawal of fluid 2 during the atomizing process. The gear 20 has sliding surfaces 21 on the upper shell 16 and/or on the holder 6, which can make holder 6 move axially when the holder 6 is rotated relative to the upper shell 16.

In an embodiment, the holder 6 is not blocked for too long and can perform the major shifting. Therefore, the fluid 2 is pushed out and atomized.

In an embodiment, when holder 6 is in the clamping position, the sliding surfaces 21 move out of engagement. Then the gear 20 releases the holder 6 for the opposite axial shift.

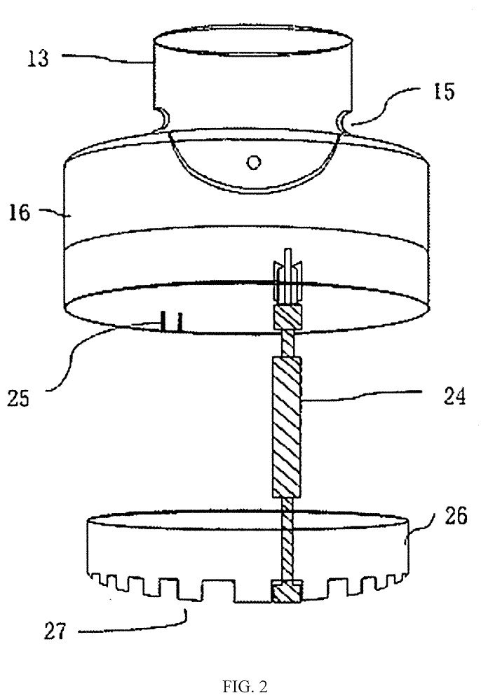

In one embodiment, the atomizer 1 includes a counter element as shown in FIG. 2. The counter element has a worm 24 and a counter ring 26. The counter ring 26 is preferably circular and has dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear so as to result in a counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. The counter can rotate relative to the lower unit of the inside part. Because of the rotation of the counter, the number displayed on the counter can change as the actuation number increases, and can be observed by the patient. After each actuation, the number displayed on the counter changes. Once the predetermined number of actuations is achieved, Protrusion A and Protrusion B will encounter each other and the counter will be prevented from further rotation. Therefore, the atomizer is blocked and stopped from further use. The number of actuations of the device can be counted by the counter.

The nebulizer described above is suitable for nebulizing the aerosol preparations according to the invention to form an aerosol suitable for inhalation. Nevertheless, the formulation according to the invention can also be nebulized using other inhalers apart from those described above, such as an ultrasonic nebulizer, a jet nebulizer, or a mesh nebulizer.

Dual therapy using a long-acting muscarinic antagonist (LAMA) and a long-acting b2-agonists (LABA) in one inhaler produces superior bronchodilation compared to monotherapy with either class alone. Bifunctional compounds combining moieties with LAMA and LABA pharmacological actions, known as MABAs, are currently in clinical development, aiming to harness the synergy between these mechanisms of action and to take advantage of a potentially simpler technical and clinical development pathway compared to LAMA/LABA combination therapies. Batefenterol is an inhaled long-acting MABA in development for the maintenance treatment of COPD.

Whereas, inhaled corticosteroids (ICS) are group of drugs such as budesonide, fluticasone furoate and mometasone furoate monohydrate, that works as, efficient local anti-inflammatory effect, it can strengthen the stability of endotheliocyte, smooth muscle cell and lysosome membrane, Immunosuppression reaction and the synthesis of reduction antibody, thus the release of the activity media such as histamine is reduced and active reduction, and can alleviate antigen-antibody in conjunction with time the enzymatic processes that excites, suppress the synthesis of bronchoconstriction material and release and alleviate the contractile response of smooth muscle. Inhaled corticosteroids drugs work by reducing inflammation, swelling, and mucus production in the airways of a person with asthma. As a result, the airways are less inflamed and less likely to react to asthma triggers, allowing people with symptoms of asthma to have better control over their condition.

Although short-acting inhaled bronchodilators (such as albuterol and ipratropium) are still used as rescue therapy, the major development has been in the introduction of long-acting inhaled β2-agonists (LABAs) and long-acting muscarinic antagonists (LAMAs), with several new products and LABA-LAMA combinations now on the market and in clinical development. New bronchodilators in development include revefenacin (TD4208), a once-daily LAMA to be delivered by nebulization, and abediterol, a once-daily LABA, in addition to existing once-daily LAMAs (tiotropium, glycopyrrolate, umeclidinium) and LABAs (indacaterol, vilanterol, olodaterol). Fixed-dose LABA-LAMA combinations for COPD include once-daily indacaterol-glycopyrrolate, vilanterol-umeclidinium, and olodaterol-tiotropium, and twice-daily formoterol-glycopyrrolate and formoterol-aclidinium. There is little difference between these drugs in terms of efficacy and safety, but they are delivered using different inhaler devices.

Several twice-daily fixed-dose inhaled corticosteroid (ICS)-LABA combinations are now on the market for asthma and COPD maintenance therapy, including fluticasone propionate-salmeterol, budesonide-formoterol, beclomethasone dipropionate-formoterol, and mometasone-formoterol, with a one once-daily combination of fluticasone furoate-vilanterol and another (mometasone-indacaterol) in development.

Fixed combinations of ICS-LABA-LAMA are now in development for treating COPD and severe asthma. One of the first triple inhalers with beclomethasone dipropionate-formoterol-glycopyrrolate for twice daily administration shows a clinical advantage over the ICS-LABA combination in patients with COPD. Several others, including once-daily fluticasone furoate-vilanterol-umeclidinium and mometasone-indacaterol-glycopyrrolate, as well as twice-daily budesonide-formoterol-glycopyrrolate, are currently in clinical development. Triple inhalers have the advantage of convenience and may improve adherence, but there are risks that the three components may interact chemically in the device, and the fixed doses may require several dose combinations.

Muscarinic antagonist-β2 agonists (MABAs), combining two pharmacophores connected by an inactive linker, are also in development. Several MABAs, including batefenterol (GSK961081), AZD2115, and AZD8871, are already in clinical trials. A major problem is that it is difficult to balance the LABA and LAMA activities, so that most MABAs tend to have a predominance of either LABA or LAMA activity. MABAs combined with an ICS are also in development as functional triple combinations.

### EXAMPLES

Materials and Reagents:
50% benzalkonium chloride aqueous solution purchased from Spectrum Pharmaceuticals Inc.
Edetate disodium dehydrate purchased from Merck.
Sodium hydroxide purchased from Titan reagents.
Hydrochloric acid purchased from Titan reagents.
Citric acid purchased from Merck.
Sodium chloride purchased from Titan reagents.
Batefenterol and fluticasone furoate are also commercially available and may be purchased.

### Example 1

The preparation of a solution for administration by soft mist inhalation is as follows:

50% benzalkonium chloride aqueous solution and edetate disodium dihydrate according to the amounts in Table 1 were dissolved in 90 ml of purified water and the solution adjusted to the target pH with hydrochloric acid or sodium hydroxide. Batefenterol and fluticasone furoate, according to the amounts in Table 1, were added to the solution and the resulting mixture sonicated until the components completely dissolved. Finally, purified water was added to provide a final volume of 100 ml.

TABLE 1
Ingredient of Sample I

| Ingredients | Sample I |
|---|---|
| Batefenterol Succinate | 30 g |
| Fluticasone Furoate | 19 g |
| polysorbate 80 | 30 mg |
| Edetate Disodium | 15 mg |
| Dihydrate | |
| 50% benzalkonium chloride aqueous | 30 mg |
| solution | |
| Hydrochloric acid or sodium | To pH 3-4 |
| hydroxide | |
| Purified water | Added to 100 ml |

### Example 2

The preparation of a solution for administration by soft mist inhalation is as follows:

50% benzalkonium chloride aqueous solution, edetate disodium dihydrate, and polysorbate 80, according to amounts in Table 2, were dissolved in 90 ml of purified water and the solution adjusted to the target pH with hydrochloric acid or sodium hydroxide. Batefenterol succinate and fluticasone furoate, according to amounts in Table 2, were added to the solution and the resulting mixture sonicated until the components completely dissolved. Finally, purified water was added to provide a final volume of 100 ml.

TABLE 2
Ingredient of Sample II

| Ingredients | Sample II |
|---|---|
| Batefenterol Succinate | 25 g |
| Fluticasone Furoate | 17 g |
| polysorbate 80 | 20 mg |
| Edetate Disodium | 10 mg |
| Dihydrate | |
| 50% benzalkonium chloride aqueous | 20 mg |
| solution | |
| Hydrochloric acid or sodium | To pH 3-4 |
| hydroxide | |
| Purified water | Added to 100 ml |

### Example 3

The preparation of a solution for administration by nebulization inhalation (Sample III) as follows:

NaCl, according to the amount in Table 3, was dissolved in 90 ml of purified water and the solution adjusted to the target pH with hydrochloric acid or sodium hydroxide. Batefenterol succinate and fluticasone furoate, according to the amounts in Table 3, were added to the solution and the resulting mixture sonicated until the components completely dissolved. Finally, purified water was added to provide a final volume of 100 ml.

TABLE 3
Ingredient of Sample III

| Ingredients | Sample III |
|---|---|
| Batefenterol Succinate | 7.5 g |
| Fluticasone Furoate | 2.5 g |
| NaCl | 150 mg |
| 50% benzalkonium chloride aqueous | 20 mg |
| solution | |
| Hydrochloric acid or sodium | To pH 3-4 |
| hydroxide | |
| Purified water | Added to 100 ml |

### Example 4

The preparation of a solution for administration by nebulization inhalation (sample IV) is as follows:

NaCl, according to the amounts in Table 4, was dissolved in 90 ml of purified water and the solution was adjusted to the target pH with hydrochloric acid or sodium hydroxide. Batefenterol succinate and fluticasone furoate, according to the amounts in Table 4, were added to the solution and the resulting mixture sonicated until the components completely dissolved. Finally, purified water was added to provide a final volume of 100 ml.

TABLE 4
Ingredient of Sample IV

| Ingredients | Sample IV |
|---|---|
| Batefenterol Succinate | 5 g |
| Fluticasone Furoate | 1.5 g |
| 50% benzalkonium chloride aqueous | 20 mg |
| solution | |
| NaCl | 250 mg |
| Hydrochloric acid or sodium | To pH 3-4 |
| hydroxide | |
| Purified water | Added to 100 ml |

### Example 5

Aerodynamic Particle Size Distribution:

The aerodynamic particle size distribution of Sample III in Example 3 was determined using a Next Generation Pharmaceutical Impactor (NGI).

The device is the soft mist inhaler device disclosed in US20190030268, entitled “inhalation atomizer comprising a blocking function and a counter”.

The device was held close to the NGI inlet until no aerosol was visible. The flow rate of the NGI was set to 30 L/minute and was operated under ambient temperature and a relative humidity (RH) of 90%.

The solution of Sample III was discharged into the NGI. Fractions of the dose were deposited at different stages of the NGI, in accordance with the particle size of the fraction. Each fraction was washed from the stage and analyzed using HPLC.

The result is shown in Tables 5 and 6.

TABLE 5
Aerodynamic Particle Size Distribution of Sample III in Example 3

| | Batefenterol Succinate | | Cut-off |
|---|---|---|---|
| | Dosage | Percentage content | diameters |
| Deposited | (μg) | at all levels % | at 30 L/min (μm) |
| Throat | 269.22 | 16.17 | |
| Stage 1 | 88.90 | 5.34 | 11.72 |
| Stage 2 | 249.26 | 14.97 | 6.4 |
| Stage 3 | 406.34 | 24.41 | 3.99 |
| Stage 4 | 404.49 | 24.30 | 2.30 |
| Stage 5 | 165.18 | 9.92 | 1.36 |
| Stage 6 | 47.12 | 2.83 | 0.83 |
| Stage 7 | 8.55 | 0.51 | 0.54 |
| MOC | 25.49 | 1.53 | |

| | |
|---|---|
| Theoretical dose (μg) | 1657.5 |
| Actual test dose (μg) | 1664.56 |
| Recovery rate (%) | 100.43 |
| ISM (μg) | 1306.44 |
| FPD (μg) | 1057.18 |
| FPF (%) | 63.51 |

TABLE 6
Aerodynamic Particle Size Distribution of Sample III in Example 3

| | Fluticasone Furoate | | |
|---|---|---|---|
| | Dosage | Percentage content | Cut-off diameters |
| Deposited | (μg) | at all levels % | at 30 L/min (μm) |
| Throat | 106.28 | 18.72 | |
| Stage 1 | 34.08 | 6.00 | 11.72 |
| Stage 2 | 85.91 | 15.13 | 6.4 |
| Stage 3 | 145.58 | 25.64 | 3.99 |
| Stage 4 | 128.32 | 22.60 | 2.30 |
| Stage 5 | 45.23 | 7.97 | 1.36 |
| Stage 6 | 10.60 | 1.87 | 0.83 |
| Stage 7 | 4.5 | 0.79 | 0.54 |
| MOC | 7.33 | 1.29 | |

| | |
|---|---|
| Theoretical dose (μg) | 552.5 |
| Actual test dose (μg) | 567.82 |
| Recovery rate (%) | 102.77 |
| ISM (μg) | 427.46 |
| FPD (μg) | 341.55 |
| FPF (%) | 60.15 |

MOC is Micro-Orifice Collector.
ISM is Impactor Size Mass.
FPF is Fine Particle Fraction.
FPD is fine particle dose.

### Example 6

pH Stability Profile

The preparation of a formulation for administration by nebulization inhalation (Formulations 1-4) is as follows:

NaCl, according to the amount in Table 7, was dissolved in 90 ml of purified water and the solution adjusted to the target pH with hydrochloric acid or sodium hydroxide. Batefenterol succinate and fluticasone furoate, according to the amounts in Table 7, were added to the solution and the resulting mixture sonicated until the components completely dissolved. Finally, purified water was added to provide a final volume of 100 ml.

TABLE 7
Ingredients of Formulations 1-4

| Ingredients | Formulation 1 | Formulation 2 | Formulation 3 | Formulation 4 |
|---|---|---|---|---|
| Batefenterol | 7.5 g | 7.5 g | 7.5 g | 7.5 g |
| Succinate | | | | |
| Fluticasone Furoate | 2.5 g | 2.5 g | 2.5 g | 2.5 g |
| NaCl | 150 mg | 150 mg | 150 mg | 150 mg |
| 50% benzalkonium | 20 mg | 20 mg | 20 mg | 20 mg |
| chloride aqueous | | | | |
| solution | | | | |
| Hydrochloric acid | To pH 2.0 | To pH 3.0 | To pH 4.0 | To pH 5.0 |
| or sodium | | | | |
| hydroxide | | | | |
| Purified water | Added to 100 ml | Added to 100 ml | Added to 100 ml | Added to 100 ml |

TABLE 8
Result of Accelerated Storage Condition of Formulation 1-4

| | | Accelerated Storage Condition | | | |
|---|---|---|---|---|---|
| | | (60° C. ± 2° C.) | | | |
| | | Formulation 1 | Formulation 2 | Formulation 3 | Formulation 4 |
| Time | Content | (pH 2.0) | (pH 3.0) | (pH 4.0) | (pH 5.0) |
| Initial/ | Description | Clear | Clear Colorless | Clear Colorless | Clear Colorless |
| 0 days | | Colorless | solution | solution | solution |
| | | solution | | | |
| | Single | 0.02 | 0.03 | 0.06 | 0.12 |
| | maximum | | | | |
| | impurity (% | | | | |
| | w/w) | | | | |
| | Total | 0.08 | 0.08 | 0.07 | 0.10 |
| | impurities | | | | |
| | (% w/w) | | | | |
| 7 days | Description | Clear | Clear Colorless | Clear Colorless | Clear Colorless |
| | | Colorless | solution | solution | solution |
| | | solution | | | |
| | Single | 0.14 | 0.11 | 0.15 | 0.35 |
| | maximum | | | | |
| | impurity (% | | | | |
| | w/w) | | | | |
| | Total | 0.21 | 0.25 | 0.23 | 0.47 |
| | impurities | | | | |
| | (% w/w) | | | | |
| 28 | Description | Clear | Clear Colorless | Clear Colorless | Clear Colorless |
| days | | Colorless | solution | solution | solution |
| | | solution | | | |
| | Single | 0.59 | 0.49 | 0.55 | 1.88 |
| | maximum | | | | |
| | impurity (% | | | | |
| | w/w) | | | | |

The experimental results show that the formulation is most stable when the pH is 3.

The preparation is stable when the pH ranges from about 2 to about 4.

### Example 7

Stability Studies

The stability study is for Sample I of Example 1.

TABLE 9
Accelerated and Long Term Stability

| | | | Accelerated Storage Condition | | | |
|---|---|---|---|---|---|---|
| | | | (40° C. ± 2° C./75% RH ± 5% RH) | | | |
| | | | | 2 | 3 | 6 |
| Content | Specification | Initial | 1 month | months | months | months |
| Description | Clear | Complies | Complies | Complies | Complies | Complies |
| | Colorless | | | | | |
| | solution | | | | | |
| Batefenterol | 90-110% | 99.8 | 99.6 | 99.1 | 98.7 | 97.4 |
| Succinate Content | | | | | | |
| (% w/w) | | | | | | |
| Fluticasone | 90-110% | 100.3 | 100.2 | 100.2 | 99.8 | 98.8 |
| Furoate Content (% | | | | | | |
| w/w) | | | | | | |
| EDTA Content (% | 90-110% | 100.8 | 100.7 | 100.5 | 101.1 | 98.3 |
| w/w) | | | | | | |
| BAC Content (% | 90-110% | 100.2 | 100.3 | 100.1 | 98.5 | 97.8 |
| w/w) | | | | | | |
| pH | 3-4 | 3.58 | 3.58 | 3.62 | 3.55 | 3.59 |
| Single maximum | NMT 0.5 | 0.02 | 0.09 | 0.15 | 0.26 | 0.33 |
| impurity | | | | | | |
| (% w/w) | | | | | | |
| Total impurities | NMT 1.0 | 0.08 | 0.12 | 0.18 | 0.35 | 0.49 |
| (% w/w) | | | | | | |

Long Term Storage
Condition
(25° C. ± 2° C./60%
RH ± 5% RH)

| Content | Specification | Initial | 3 months | 6 months |
|---|---|---|---|---|
| Description | Clear Colorless | Complies | Complies | Complies |
| | solution | | | |
| Batefenterol Succinate | 90-110 % | 99.8 | 99.8 | 99.1 |
| Content (% w/w) | | | | |
| Fluticasone Furoate | 90-110% | 100.3 | 100.1 | 99.3 |
| Content (% w/w) | | | | |
| EDTA Content (% w/w) | 90-110% | 100.8 | 100.7 | 98.5 |
| BAC Content (% w/w) | 90-110% | 100.2 | 100.7 | 98.5 |
| pH | 3-4 | 3.58 | 3.52 | 3.57 |
| Single maximum | NMT 0.5 | 0.02 | 0.08 | 0.19 |
| impurity (% w/w) | | | | |
| Total impurities (% w/w) | NMT 1.0 | 0.08 | 0.16 | 0.21 |

NMT is not more than.

Sample IV is stable at 40° C.±2° C./75% RH±5% RH for 6 months.

What is claimed is:

1. A propellant free pharmaceutical formulation comprising batefenterol or a pharmaceutically acceptable salt thereof, fluticasone furoate, a surfactant, and a pH adjusting agent.

2. The pharmaceutical formulation of claim 1, wherein the batefenterol or a pharmaceutically acceptable salt thereof is batefenterol succinate.

3. The pharmaceutical formulation of claim 2, wherein the batefenterol succinate is present in an amount ranging from about 1 mg/ml to about 200 mg/ml.

4. The pharmaceutical formulation of claim 1, wherein the fluticasone furoate is present in an amount ranging from about 100 mg/100 ml to about 20 g/100 ml.

5. The pharmaceutical formulation of claim 1, wherein the surfactant is present in an amount ranging from about Omg/100 ml to about 100 mg/100 ml.

6. The pharmaceutical formulation of claim 1, wherein the surfactant is a polysorbate.

7. The pharmaceutical formulation of claim 1, wherein the surfactant is a polysorbate 80.

8. The pharmaceutical formulation of claim 1, wherein the pH ranges from about 2.0 to about 6.0.

9. The pharmaceutical formulation of claim 1, further comprising a preservative.

10. The pharmaceutical formulation of claim 1, wherein the preservative is selected from the group consisting of benzalkonium chloride, benzoic acid, and sodium benzoate.

11. The pharmaceutical formulation of claim 10, wherein the preservative is present in an amount ranging from about 2 mg/100 ml to about 300 mg/100 ml.

12. The pharmaceutical formulation of claim 10, wherein the preservative is benzalkonium chloride in an amount of about 10 mg/100 ml.

13. The pharmaceutical formulation of claim 1 further comprising a complexing agent selected from the group consisting of edetic acid (EDTA), disodium edetate, and edetate disodium dihydrate, wherein the complexing agent is present in an amount ranging from about 1 mg/100 ml to about 500 mg/100 ml.

14. A method for administering the pharmaceutical formulation of claim 1 comprising nebulizing a defined amount of the pharmaceutical formulation with an inhaler by using pressure to force the pharmaceutical preparation through a nozzle to form an inhalable aerosol.

15. The method of claim 14, wherein the inhalable aerosol has an average particle size of less than about 15 microns.

16. The method of claim 14, wherein the defined amount of the pharmaceutical formulation is less than about 70 microliters.

17. A method of treating chronic obstructive pulmonary disease (COPD) or asthma in a patient comprising administering the formulation of claim 1 to the patient by inhalation.

18. The pharmaceutical formulation of claim 1 comprising:
an aqueous solution comprising:
(i) batefenterol succinate in an amount of about 30 g/100 ml,
(ii) fluticasone furoate in an amount of about 19 g/100 ml,
(iii) polysorbate 80 in an amount of about 30 mg/100 ml,
(iv) edetate disodium dihydrate in an amount of about 15 mg/100 ml,
(v) 50% benzalkonium chloride aqueous solution in an amount of about 30 mg/100 ml, wherein the pH of the formulation ranges from about 3 to about 4.

19. The pharmaceutical formulation of claim 1 comprising:
an aqueous solution comprising:
(i) batefenterol succinate in an amount of about 25 g/100 ml,
(ii) fluticasone furoate in an amount of about 17 g/100 ml,
(iii) polysorbate 80 in an amount of about 20 mg/100 ml,
(iv) edetate disodium dihydrate in an amount of about 10 mg/100 ml,
(v) 50% benzalkonium chloride aqueous solution in an amount of about 20 mg/100 ml, wherein the pH of the formulation ranges from about 3 to about 4.

20. A pharmaceutical formulation comprising:
an aqueous solution comprising:
(i) batefenterol succinate in an amount of about 7.5 g/100 ml,
(ii) fluticasone furoate in an amount of about 2.5 g/100 ml,
(iii) sodium chloride in an amount of about 150 mg/100 ml,
(iv) 50% benzalkonium chloride aqueous solution in an amount of about 20 mg/100 ml, wherein the pH of the formulation ranges from about 3 to about 4.

21. A pharmaceutical formulation comprising:
an aqueous solution comprising:
(i) batefenterol succinate in an amount of about 5 g/100 ml,
(ii) fluticasone furoate in an amount of about 1.5 g/100 ml,
(iii) sodium chloride in an amount of about 250 mg/100 ml,
(iv) 50% benzalkonium chloride aqueous solution in an amount of about 20 mg/100 ml, wherein the pH of the formulation ranges from about 3 to about 4.

22. The method of claim 17, wherein the batefenterol succinate is administered at a daily dose ranging from about 50 micrograms to about 1000 microgram and the fluticasone furoate is administered at a daily dose ranging from about 1 microgram to about 1000 micrograms.
